(19) United States
(12) Patent Application Publication
(10) Pub. No.: US 2022/0296415 A1
(43) Pub. Date: Sep. 22, 2022

(54) SUCTION FORCEPS

(71) Applicants: Tarek Hassan, Ann Arbor, MI (US); Omar T. Hassan, Ann Arbor, MI (US)

(72) Inventors: Tarek Hassan, Ann Arbor, MI (US); Omar T. Hassan, Ann Arbor, MI (US)

(21) Appl. No.: 17/699,475

(22) Filed: Mar. 21, 2022

Related U.S. Application Data
(60) Provisional application No. 63/164,035, filed on Mar. 22, 2021.

(51) Int. Cl. A61F 9/007

(52) U.S. Cl. CPC A61F 9/00736

(57) ABSTRACT

Microsurgical suction forceps are well suited to intraocular procedures including macular and WM peeling. At least one hollow member includes a jaw surface with one or more micromachined apertures in communication with a source of suction having a suction pressure that is sufficient to enhance the adhesion of tissue against the first jaw surface, but insufficient to intentionally draw the tissue into the apertures. The jaw surfaces may be elongated, having a length that exceeds their width, or the jaw surfaces may be disposed on the distal ends of members that are bent outwardly then toward one another such that the opposing jaw surfaces form a pincer configuration. The apertures are laser-drilled or otherwise micromachined, and at least the distal ends of the members may be constructed in piecewise fashion and joined together. The forceps may be disposable and provided in sterile packaging ready for use. Methods of use are also disclosed.

### REFERENCE TO RELATED APPLICATIONS

This application claims priority to and the benefit of U.S. Provisional Patent Application Ser. No. 63/164,035, filed Mar. 22, 2021, the entire content of which is incorporated herein by reference.

### FIELD OF THE INVENTION

This invention relates generally to surgical forceps and, in particular, to microsurgical forceps and tissue grasping instruments with distal suctioning to enhance tissue adherence and/or prevent tissue loss.

### BACKGROUND OF THE INVENTION

There are extremely delicate microsurgical operations wherein minute pieces of tissue are grasped and removed to treat a particular condition. For example, one such procedure, macular peeling, refers to the surgical technique used to remove epiretinal fibrous tissue or the internal limiting membrane (ILM) in the macula of the eye. Macular or ILM peeling may be used to treat various vitreoretinal disorders, including macular holes, macular puckers, epiretinal membranes (ERMs), diabetic macular edema, retinal detachment, proliferative vitreoretinopathy, retinal vein occlusions, vitreomacular traction, optic pit maculopathy, and Terson syndrome.

In accordance with a typical macular peeling procedure, an initial flap is created in the epiretinal fibrous tissue and/or ILM with a sharp instrument such as pick forceps, bent MVR (micro-vitreoretinal) instrument, or vitreoretinal forceps. Once the flap is created, the desired area of ERM and/or ILM is removed with the vitreoretinal forceps using a variety of circular movements around the fovea similar to a capsulorhexis and in parallel to the retinal surface. The extent of surface membrane tissue to be peeled varies from limited regions centered at the fovea to areas extending from the superotemporal to the inferotemporal vascular arcades. Staining of the ILM with adjuvant dyes can make the procedure easier to perform and more effective, reducing also the operating time and the mechanical trauma to the retina. Such peeling procedures are carried out in conjunction with a vitrectomy.

ILM peeling has evolved in the last two decades, and the different instrumentations and approaches have progressively advanced such procedures into a safer, easier, and more useful option for the vitreoretinal surgeon. There are now many different types of membrane peeling forceps with varying sizes, shapes, and compositions. All have the ability to grasp a membrane only with the tip of forceps, and hence such instruments also called ‘end-gripping,’ ‘end-grabbing,’ or ‘epiretinal forceps’. Some tools are engineered for peeling and grasping, and some are diamond dusted to prevent membrane slippage from the forceps. Power-driven models are also available.

Despite advances, complications arise due to the delicate nature of the procedure. A membrane peel with forceps must be capable of holding a very thin ILM (approximately 2.5 microns thick) while being pulled away and removed from the macula, and then removed from the eye. This is tedious and time-consuming, and occasionally the membrane shreds. There is an outstanding need for improved instrumentation and procedures to assist with holding the membrane in place during peeling.

### SUMMARY OF THE INVENTION

This invention resides in microsurgical suction forceps and methods of use. The invention is particularly suited to intraocular procedures of the type discussed in the Background of the Invention, including macular and ILM peeling procedures and all other surface epiretinal fibrous tissue. Forceps constructed in accordance with the invention comprise two elongated members with proximal and distal ends, including a first member terminating in a distal end with a first jaw surface, and a second member terminating in a distal end with a second jaw surface in opposing relation to the first jaw surface. The members are disposed in an outer sheath with the distal ends protruding from a distal opening in the sheath, such that when the two elongated members are drawn proximally into the sheath, the jaw surfaces are brought together to grasp tissue.

In accordance with the invention, at least the first member comprises a hollow tube, and wherein the first jaw surface includes one or more micromachined apertures in communication with the hollow tube of the first member. The proximal end of the first member is in communication with a source of suction having a suction pressure that is sufficient to enhance the adhesion of tissue against the first jaw surface, but likely be insufficient to intentionally draw the tissue into the apertures.

In preferred embodiments, the surfaces are configured and dimensioned for an intraocular peeling procedure such as a macular or epiretinal membrane peeling procedure wherein preretinal tissue, a portion of an internal limiting membrane (ILM) in the macula of an eye, or even retinal tissue itself is removed. The jaw surfaces may be elongated, having a length that exceeds their width, or the jaw surfaces may be disposed on the distal ends of members that are bent outwardly then toward one another such that the opposing jaw surfaces form a pincer configuration.

In some embodiments the apertures are laser-drilled apertures, and at least the distal ends of the members may be constructed in piecewise fashion and joined together. In some embodiments both members are hollow, and both jaw surfaces contain apertures that could be created by other drilling methods. In preferred embodiments the forceps are disposable and provided in sterile packaging ready for use. Methods of performing a microsurgical procedures using the inventive forceps are also disclosed herein.

### BRIEF DESCRIPTION OF THE DRAWINGS

FIG. 1 is a perspective view of dual-grasping forceps according to the invention;

FIG. 2 is a side view of the dual-grasping forceps of FIG. 1;

FIG. 3 is a cross-sectional view of the dual-grasping forceps of FIG. 1;

FIG. 4 a drawing of an alternative embodiment of the invention, wherein the apertures of the grasping surface are laser drilled or otherwise mechanically formed in the body of the jaw(s);

FIG. 5 is a cross-sectional view of the dual-grasping forceps of FIG. 4;

FIG. 6 shows a jaw with micro-machined apertures;

FIG. 7 is a perspective view of a further alternative embodiment of the invention; namely, a pincer-style design wherein the jaws include very small opposing gripping surfaces;

FIG. 8 is a side view of the pincer forceps of FIG. 7;

FIG. 9 is a cross-sectional view of the pincer forceps of FIG. 7;

FIG. 10 is a perspective view of a single pincer jaw tip; and

FIG. 11 is a cross section showing jaw members entering into a sheath along with suction conduits.

### DETAILED DESCRIPTION OF THE INVENTION

This invention improves upon microsurgical forceps by providing aspiration through the small openings in one or both of the jaws of the instrument to achieve a more reliable hold on tissue during peeling and removal. The broad concept does not anticipate the use strong aspiration for direct auction of the tissue, but rather, to use delicate suction for the purpose of holding tissue to be removed in the jaws of the forceps. In simpler terms, the invention uses suction for additional mechanical leverage—to enhance holding power—as opposed to the use of suction through a conduit for regional evacuation.

The invention is not limited in terms of the particular grasping instrument or source of suction; however, the invention is ideally suited to vitreoretinal surgical procedures, with suction coming from an aspiration line connected to a vitrector machine, for example. Even within the realm of vitreoretinal surgery, the invention is applicable to multiple different types and styles of forceps, including yet-to-be developed designs, regardless of jaw geometry, with suction on one or both jaws of the instrument.

The inventive forceps described herein may be shaped in accordance with existing forceps, with the exception that one or more apertures are provided on one or both of the grasping surface. Such apertures are in communication with minute tubing or cannula to a source of gaseous or fluidic suction, with the number and size of the apertures being dictated by the type of forceps, which in turn dictates the size and shape of the grasping surface.

In preferred embodiments, the instruments are disposable, with the jaw surfaces of the forceps defining an area of less than 10 mm, and with the apertures being formed with a precise machining process capable of forming very small, precise holes. The areas or diameters of the apertures are typically less than 1 millimeter, more preferably down to 1 micron in some cases. In a conventional intraocular peeling procedure, the jaw surfaces might be less that 5 mm, and the apertures would be in the 1-100 micron range. In some embodiments, laser drilling is used to form the apertures though other techniques can be envisioned to create them as well. Note that while the drawings show certain jaw geometries with a certain number of apertures, more or fewer apertures may be provided in each instance.

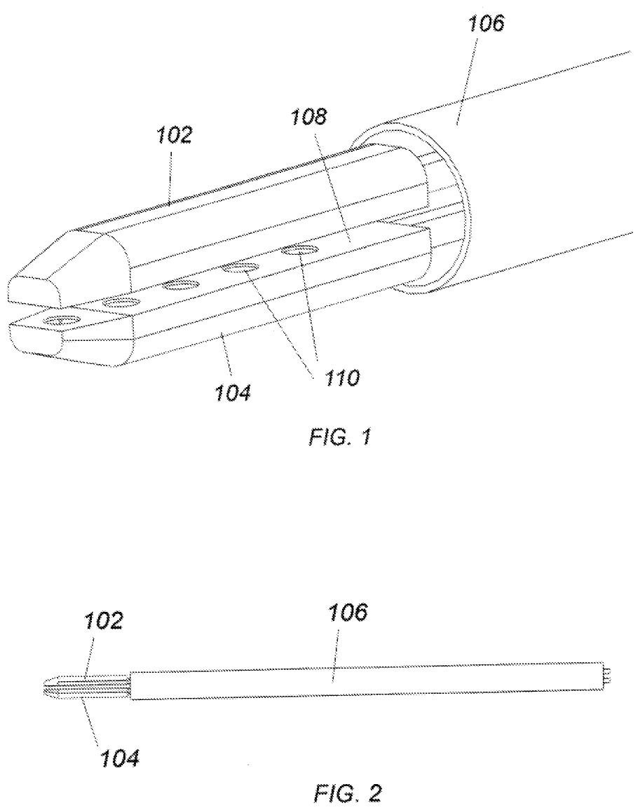

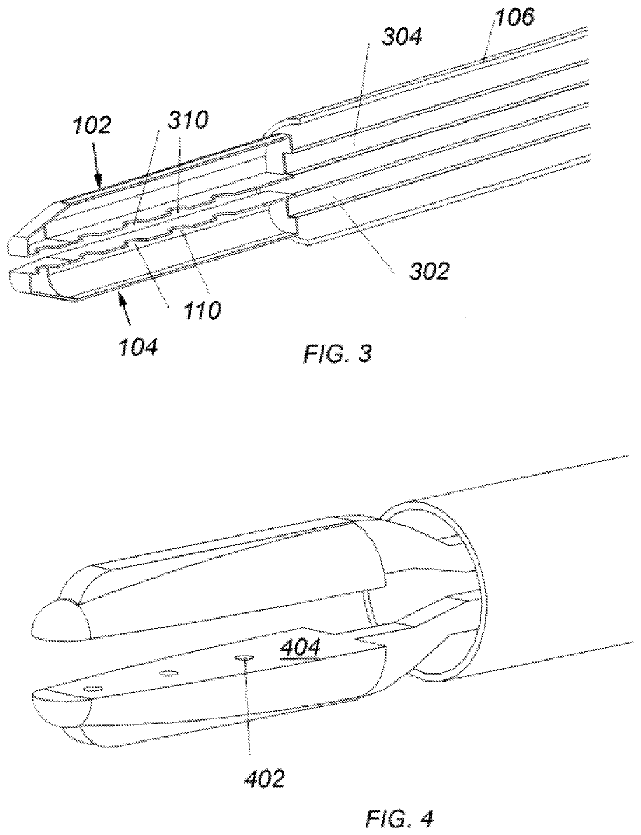

FIGS. 1-3 illustrate a dual-grasping forceps applicable to the invention. FIG. 1 is a perspective view; FIG. 2 is side view; and FIG. 3 is a cross section. In this particular embodiment, opposing jaws 102, 104 are retracted into sheath 106, causing the jaws to close with a substantially parallel alignment. Minute apertures 110 are seen on the grasping surface 108, with the understanding that one or both of the grasping surfaces may include such apertures. The cross section of FIG. 3 shows that the jaws 102, 104 may be implemented as hollow structures, such that the apertures 110, 310 (now seen on both grasping surfaces) are able to communicate with cavities 302, 304 leading to a source of suction. FIG. 3 also shows one way in which the jaws may be fabricated; namely, by joining one or two pieces to form a hollow member, which the apertures being formed before or after joining.

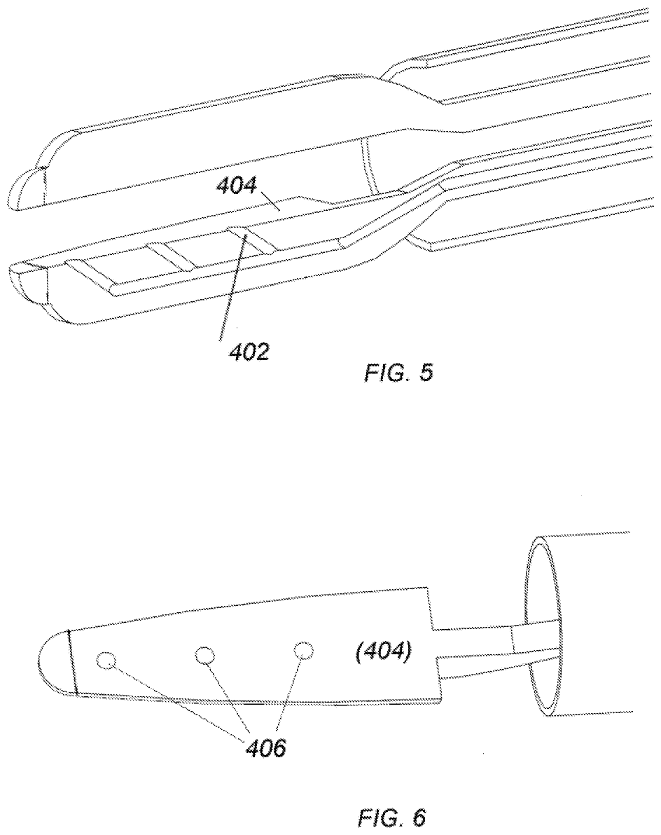

FIGS. 4-6 show a different style of forceps, wherein the apertures 402 of grasping surface 404 are actually laser drilled or otherwise mechanically formed in the body of the jaw(s), as seen in the cross sectional view of FIG. 5. While the drawing shows suction being provided on one side, both jaws may be so modified.

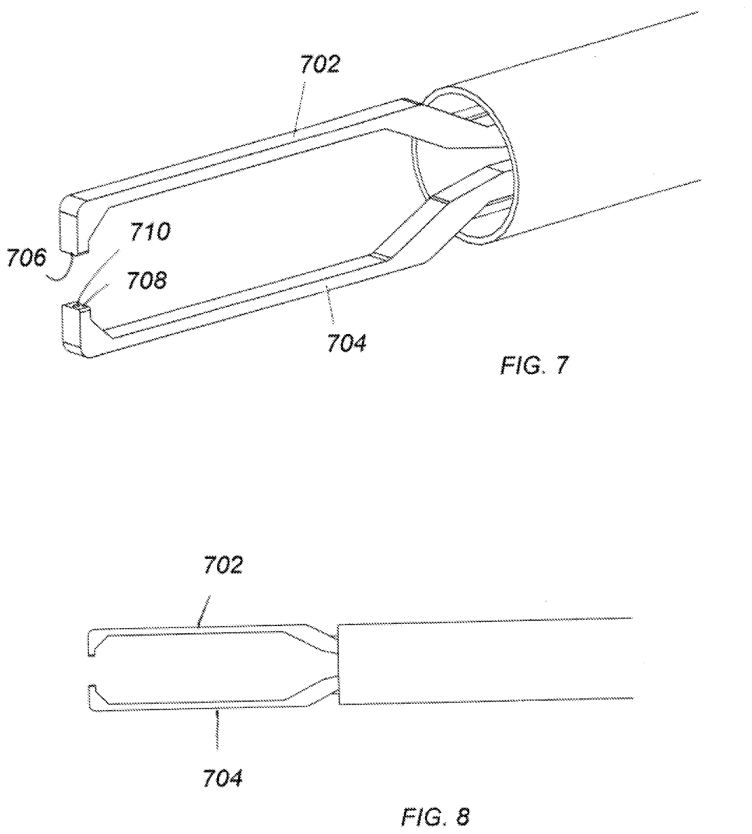

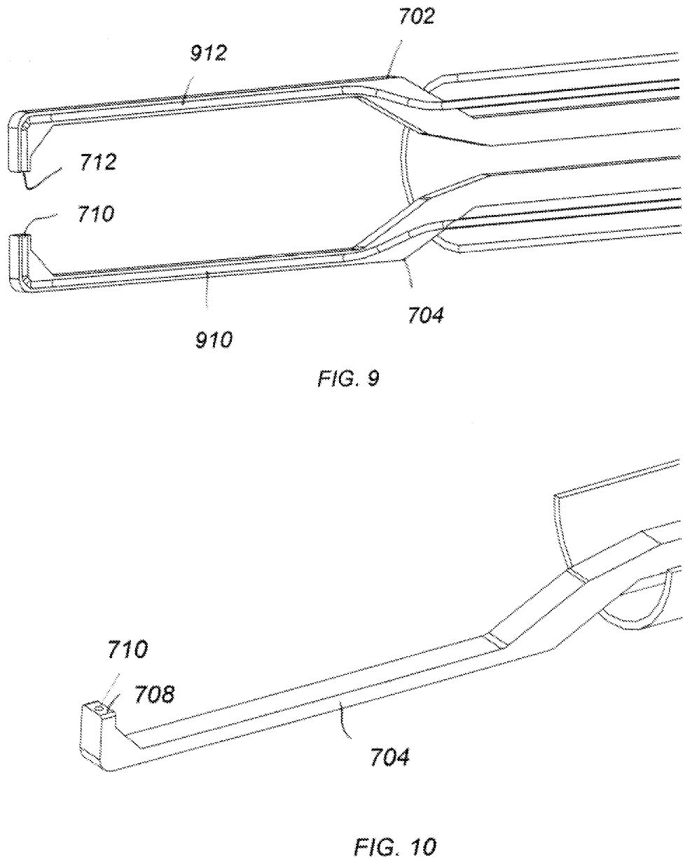

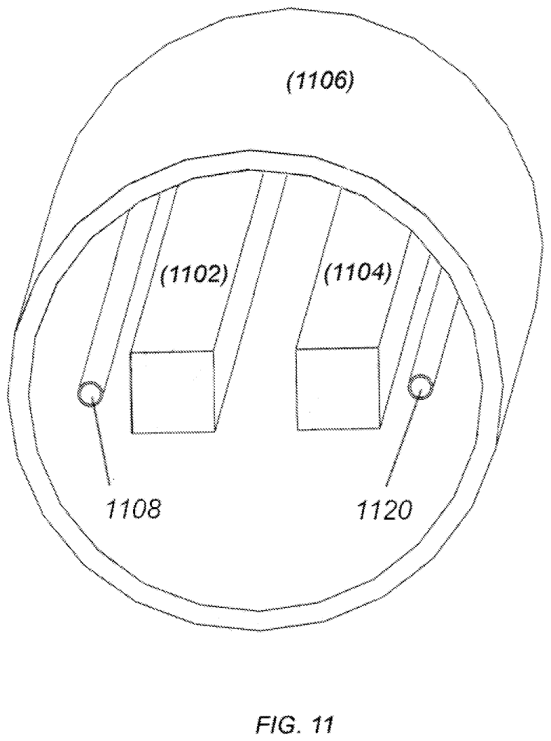

FIG. 7-10 illustrate yet a further type of forceps to which the invention is applicable, in this case a pincer-style design wherein the jaws 702, 704 include very small opposing gripping surfaces 706, 708. Such a physical configuration limits the number of possible aperture locations to a few, with a single aperture 710 being shown on surface 708 of jaw 704, again with the understanding that one or more opposing apertures may be present on surface 706 of jaw 702. The cross section of FIG. 9 shows the way in which apertures 710, 712 communicate with suction conduits 910, 912. While the drawing shows the conduits being mechanically drilled, as with other embodiments, including the one depicted in FIGS. 1-3, the body of the jaws may be hollow. FIG. 11 is a cross section showing jaw members 1102, 1104 entering into sheath 1106 along with suction conduits 1108, 1110.

1. Microsurgical forceps, comprising:
two elongated members with proximal and distal ends, including a first member terminating in a distal end with a first jaw surface, and a second member terminating in a distal end with a second jaw surface in opposing relation to the first jaw surface;
wherein the members are disposed in an outer sheath with the distal ends protruding from a distal opening in the sheath, such that when the two elongated members are drawn proximally into the sheath, the jaw surfaces are brought together to grasp tissue;
wherein at least the first member comprises a hollow tube, and wherein the first jaw surface includes one or more micromachined apertures in communication with the hollow tube of the first member;
wherein the proximal end of the first member is in communication with a source of suction having a suction pressure; and
wherein the suction pressure is sufficient to enhance the adhesion of tissue against the first jaw surface, but insufficient to intentionally draw the tissue into the apertures.

2. The microsurgical forceps of claim 1, wherein the surfaces are configured and dimensioned for an intraocular peeling procedure.

3. The microsurgical forceps of claim 1, wherein the surfaces are configured and dimensioned for a macular or membrane peeling procedure.

4. The microsurgical forceps of claim 1, wherein the tissue is a preretinal fibrous tissue membrane or retinal tissue itself.

5. The microsurgical forceps of claim 1, wherein the tissue is an internal limiting membrane (ILM) in the macula of an eye.

6. The microsurgical forceps of claim 1, wherein the jaw surfaces are elongated, having a length that exceeds their width.

7. The microsurgical forceps of claim 1, wherein the jaw surfaces are distal ends of the two members are bent outwardly then toward one another such that the opposing jaw surfaces form a pincer configuration.

8. The microsurgical forceps of claim 1, wherein the apertures are laser-drilled apertures.

9. The microsurgical forceps of claim 1, wherein at least the distal ends of the members are constructed as joined pieces.

10. The microsurgical forceps of claim 1, wherein both members are hollow and both jaw surfaces contain apertures in communication with respective hollow members.

11. A method of performing a microsurgical procedure, comprising the steps of:
(a) providing the forceps of claim 1;
(b) inserting the distal ends of the forceps into an intraocular space;
(c) grasping tissue with the jaw surfaces with
(d) removing the tissue from the intraocular space; and
(e) repeating steps (b)-(d) until the tissue is adequately removed.

12. The method of claim 11, wherein steps (b)-(d) are associated with an intraocular peeling procedure.

13. The method of claim 11, wherein steps (b)-(d) are associated with an epiretinal macular membrane peeling procedure.

14. The method of claim 11, wherein the tissue is retinal tissue itself.

15. The method of claim 11, wherein the tissue is extramacular preretinal tissue.

16. The method of claim 11, wherein the tissue is an internal limiting membrane (ILM) in the macula of an eye.

17. The method of claim 11, wherein the material removed from the eye is a foreign body.

18. The method of claim 11, wherein the tissue is inflammatory debris and/or membranes.

19. The microsurgical forceps of claim 1, wherein the forceps are disposable.
